(19) United States
(12) Patent Application Publication
(10) Pub. No.: US 2021/0087129 A1
(43) Pub. Date: Mar. 25, 2021

(54) PROCESS FOR MAKING ETHYLENE GLYCOL AND/OR PROPYLENE GLYCOL FROM ALDOSE- AND/OR KETOSE-YIELDING CARBOHYDRATES WITH EX SITU HYDROGENOLYSIS OR HYDROGENATION CATALYST TREATMENT

(71) Applicant: Iowa Corn Promotion Board, Johnston, IA (US)

(72) Inventors: David James Schreck, Lake City, MN (US); Donald Bunning, South Charleston, WV (US); Lou Kapicak, Cross Lanes, WV (US); Brooke Albin, Charleston, WV (US); Mark Nunley, Charleston, WV (US); Michael Bradford, Charleston, WV (US)

(21) Appl. No.: 17/031,675

(22) Filed: Sep. 24, 2020

Related U.S. Application Data
(60) Provisional application No. 62/904,854, filed on Sep. 24, 2019.

(51) Int. Cl. C07C 29/17; B01J 38/68

(52) U.S. Cl. CPC C07C 29/172; B01J 38/68

(57) ABSTRACT

Processes are disclosed for the catalytic conversion using a heterogeneous hydrogenolysis or hydrogenation catalyst of carbohydrate feed to one or both of ethylene glycol and propylene glycol. In the disclosed processes, a portion of the heterogeneous catalyst in the reaction zone of the catalytic process is withdrawn and recycled and the recycle is integrated to enhance the overall process.

### CROSS-REFERENCE TO RELATED APPLICATION(S)

This application claims the benefit under 35 U.S.C. § 119(e) to U.S. Provisional Application 62/904,854, filed Sep. 24, 2019, and entitled “PROCESS WITH INTEGRATED RECYCLE FOR MAKING ETHYLENE GLYCOL AND/OR PROPYLENE GLYCOL FROM ALDOSE- AND/OR KETOSE-YIELDING CARBOHYDRATES,” which is hereby incorporated herein by reference in its entirety for all purposes.

### TECHNICAL FIELD

This invention pertains to processes for the catalytic production of ethylene glycol and/or propylene glycol from aldose- and/or ketose-yielding carbohydrates, particularly processes that have an integrated, ex situ treatment of hydrogenolysis or hydrogenation catalyst.

### BACKGROUND

Ethylene glycol and propylene glycol are valuable commodity chemicals and each has a broad range of uses. These chemicals are currently made from starting materials based upon fossil hydrocarbons (petrochemical routes).

Proposals have been made to manufacture ethylene glycol and propylene glycol from renewable resources such as carbohydrates, e.g., sugars. One such route has been practiced commercially and involves the fermentation of sugars to ethanol, catalytically dehydrogenating the ethanol to ethylene and the ethylene is then catalytically converted to ethylene oxide which can then be reacted with water to produce ethylene glycol. This route is not economically attractive as three conversion steps are required, and it suffers from conversion efficiency losses. For instance, the theoretical yield of ethanol is 0.51 grams per gram of sugar with, on a theoretical basis, one mole of carbon dioxide being generated per mole of ethanol.

Alternative processes to make ethylene glycol and propylene glycol from renewable resources are thus sought. These alternative processes include catalytic routes such as hydrogenolysis of sugar and a two-catalyst process using a retro-aldol catalyst to generate intermediates from sugar that can be hydrogenated over a hydrogenation catalyst to produce ethylene glycol and propylene glycol. The former process is referred to herein as the hydrogenolysis process or route, and the latter process is referred to as the hydrogenation, or retro-aldol, process or route. For the sake of ease of reference, the latter is herein referred to as the retro-aldol process or route. The term “catalytic process” or “catalytic route” is intended to encompass both hydrogenolysis and the retro-aldol route. The term “Hcat” as used herein is intended to encompass both hydrogenolysis catalysts and hydrogenation catalysts.

In the catalytic routes, carbohydrate (which may be one carbohydrate or a mixture of carbohydrates) that yields aldose or ketose, is passed to a reaction zone containing catalyst in an aqueous medium. At elevated temperature and the presence of hydrogen, the carbohydrate is converted to ethylene glycol and/or propylene glycol. The hydrogenolysis process uses a hydrogenolysis catalyst, and typically temperatures below about 225° C. In many instances, high conversions of the carbohydrate can occur at temperatures below about 220° C. The hydrogenolysis route often uses a low concentration of carbohydrate fed to the reaction zone to attenuate the production of by-products. The retro-aldol route is fundamentally different in that the carbohydrate is converted over a retro-aldol catalyst to intermediates, and then the intermediates are then catalytically converted over a hydrogenation catalyst to ethylene glycol and/or propylene glycol. The sought initially-occurring retro-aldol reaction is endothermic and requires a high temperature, e.g., often over 230° C., to provide a sufficient reaction rate to preferentially favor the conversion of carbohydrate to intermediates over the hydrogenation of carbohydrate to polyol such as sorbitol.

Over time, laboratory-scale, catalytic processes to convert carbohydrates to ethylene glycol and propylene glycol, and especially the retro-aldol route, have evidenced improvements in selectivity and conversion efficiency. These improvements have now given cause to consider the manner in which the catalytic routes should be implemented to provide a commercial-scale facility that could be competitive with the petrochemical routes to make these chemicals. It is important to maximize the use of the feedstock for valuable product, but also changes in the rates of generation of by products and coproducts should be minimized such that equipment for their removal from the sought products does not require significant turn-up or turn-down capabilities.

Deactivation of the Hcat over time is expected as is deactivation of any catalyst. The deactivation of the Hcat affects the catalytic activity density in the reactor leading both to a loss of conversion and a potential change in product slate. Hcat could be continuously or intermittently replaced, but the cost of production would increase.

Both catalytic routes, by their very nature, present a myriad of complexities that affect the economics of a commercial facility, both in capital and operating expenses. Accordingly, a desire exists to develop catalytic processes that can be cost-effective on a commercial-scale.

### BRIEF SUMMARY

By this invention, it has been found that the loss of activity of hydrogenolysis catalyst or hydrogenation catalyst is able to be attenuated or reversed (“rejuvenated”) by ex situ treatment of the catalyst. Thus, catalytic processes are provided that can enhance the economics of producing ethylene glycol and/or propylene glycol from carbohydrates. In the processes of this invention, Hcat is withdrawn from the reaction zone and treated ex situ to restore or enhance the activity of the catalyst, and then the treated Hcat is returned to the reaction zone. The catalyst is treated to maintain the selectivity to the sought products without unduly adverse changes to the production rate of undesired by products and coproducts. In some instances, the treatment will comprise attenuating the activity of the Hcat as the rejuvenated catalyst, and any fresh make-up Hcat, upon being introduced into the reaction zone can, if too active, adversely affect the product slate.

Without wishing to be limited to theory, it is believed that the concentration, dispersion and activity of the Hcat in the reaction zone affects the selectivity to the sought products, i.e., ethylene glycol and propylene glycol (referred to individually and jointly herein as “lower glycols”) and the generation of by-products. For example, with a reaction zone that has too high of a hydrogenation activity, lower glycols can be lost to further reactions including hydrogenation. It is also possible that larger, localized regions of catalytic sites can lead to hydrogen starvation, that is where the mass transfer of hydrogen to the catalytic sites is insufficient and undesired products, such as acids, are generated. With too low of an activity, feedstock is not consumed or, more likely, competitive non-catalytic reactions or, especially in the case of the retro-aldol process, competitive catalytic reactions occur that reduce the selectivity to lower glycols. The dispersion of the Hcat can also affect the product slate even though the catalyst is highly active as the mean mass transfer distance can also allow competitive non-catalytic and catalytic reactions to occur.

Again, without wishing to be limited to theory, it is believed that deactivation of the Hcat occurs from particle growth of the catalytic metals and a loss of catalytic metals, e.g., through loss of support or solubilization of the catalytic metals. By this invention, it is found that loss of catalytic activity in the reaction environment generating lower glycols can also be due to one or more phenomenon that can be remediated by ex situ treatments of the Hcat to provide rejuvenated Hcat. Without limitation, these mechanisms for deactivation can include one or more of oxidation of the catalytic metal or other selective poisoning, deposition on the Hcat of heavy organics generated as a side product and deposition of inorganics or organometallic compounds, especially in the retro-aldol process, on the Hcat. The source of the inorganics can, for instance, be pH and other adjuvants used in the process, corrosion, disintegration of the support of the Hcat, solubilization and precipitation of metals used in the Hcat and organometallics from these inorganics.

In accordance with the instantly disclosed processes, a portion of the hydrogenolysis or hydrogenation catalyst, as the case may be, is continuously or intermittently withdrawn from the reaction zone and subjected to an ex situ treatment to modulate the activity of the catalyst. The treatment can be to restore activity or to enhance performance to a sought level. In some instances, the treatment can be, or include a step, to reduce catalytic activity to a desired level. At least a portion of the treated catalyst, with or without the addition of more hydrogenolysis or hydrogenation catalyst, is returned to the reaction zone.

The catalytic process disclosed herein is effected in a reactor that contains hydrogenolysis or hydrogenation catalyst that is suspended in a solvent, typically an aqueous solvent. The term “suspended” as used herein includes any spatial arrangement of the catalyst as long as it can be moved with the movement of the surrounding liquid medium. A portion of the Hcat is withdrawn from the reactor. The Hcat is subjected to one or more unit operations to enhance the performance of the catalyst and at least a portion of the catalyst is recycled to the reactor. The term “enhanced performance” as used herein is intended to encompass any treatment of the Hcat that provides a beneficial result to the catalytic conversion and include, without limitation, increase in catalytic hydrogenation activity; increase in catalyst selectivity; modulation of catalytic hydrogenation activity; stabilization of catalytic metals on the catalyst; addition of promoters, modifiers and other adjuvants to the catalyst; and modification of the porosity of the catalyst.

The invention broadly pertains to catalytic processes for producing a lower glycol comprising at least one of ethylene glycol and propylene glycol from a carbohydrate-containing feed that comprises at least one of aldose- and ketose-yielding carbohydrate, said processes comprising continuously or intermittently supplying the feed to a reaction zone containing a liquid medium having therein one or more catalysts for converting said carbohydrate to said glycol, wherein at least one of the catalysts is a hydrogenolysis or hydrogenation catalyst that is suspended in the liquid medium, said liquid medium being at catalytic conversion conditions including the presence of dissolved hydrogen, to produce a reaction product containing said lower glycol, and continuously or intermittently withdrawing liquid medium that contains reaction product from the reaction zone, wherein
(i) continuously or intermittently at least a portion of said suspended catalyst is withdrawn from the reaction zone;
(ii) at least a portion of the withdrawn suspended catalyst is subjected to treatment to provide a treated catalyst having enhanced performance; and
(iii) at least a portion of the treated catalyst is passed to the reaction zone.

If desired, make-up or fresh catalyst (hydrogenolysis catalyst for the hydrogenolysis route or at least one of retro-aldol catalyst and hydrogenation catalyst for the retro-aldol route) for the catalytic processes can be introduced directly or indirectly into the reaction zone, for instance, by one or more of admixing with the recycle liquid phase prior to its introduction into the reaction zone or by admixing with the feed prior to its introduction into the reaction zone. In some instances, the make-up or fresh catalyst is admixed with catalyst to be treated in one or more unit operations before being introduced into the reaction zone.

The catalyst can be withdrawn from the reaction zone with the liquid medium or can be separated from at least a portion of the liquid medium as it is withdrawn, e.g., by any suitable liquid/solid separation technique including, but not limited to, filtration, hydrocyclone separation, centrifugation, vane separation, and settling. Where withdrawn with the liquid medium, advantageously at least a portion of the liquid medium is passed to the reaction zone with the treated catalyst. The portion of the withdrawn liquid medium that is recycled to the reaction zone can be an aliquot or aliquant portion. Where an aliquant portion, that is the concentration of components in the portion of the liquid medium being recycled is different from that of the withdrawn liquid medium. Aliquant portions would occur when the liquid medium withdrawn is subjected to vapor/liquid separation, a filtration, sorption or other unit operation that selectively reduces concentration of one or more components of the liquid medium as withdrawn from the reaction zone.

In one embodiment, the withdrawn liquid medium is not subjected to selective separation unit operations, but rather serves to enable other continuous or intermittent unit operations to occur outside the reaction zone. In another embodiment the withdrawn liquid medium is subjected to one or more selective separation unit operations to provide a retained liquid phase and at least a portion of the retained liquid phase is recycled to the reaction zone.

The retained liquid phase contains catalyst and can be treated to enhance performance of the catalyst. Where the active catalytic metal in the Hcat becomes oxidized, a hydrogen treatment can be conducted under conditions to reduce at least a portion of the oxidized metal to the elemental state which in turn attenuates loss of catalytic activity. An additional benefit can be realized where the oxidized form of the catalytic metal forms an ion that is soluble in the retained liquid medium, the hydrogen treatment, by reducing the portion of catalytic metal that is ionized can facilitate its recovery and attenuate loss of catalytic metal.

The heterogeneous hydrogenolysis catalyst or hydrogenation catalyst can, additionally or alternatively, be subjected to conditions to enhance the activity of the catalyst other than by hydrogen treatment as discussed above. For instance, organic or inorganic deposits on the Hcat can be removed by, e.g., extraction or chemical reaction. This unit operation can be followed by a separation unit operation to remove the extractant or reactants and products. Alternatively, the Hcat can be removed from the retained liquid medium, treated to remove organic and/or inorganic deposits, washed and then recombined with the retained liquid medium for recycle to the reaction zone. The treatment can be, for example and not by limitation, water washing, washing in an alkaline environment, washing in an acidic environment, washing in an organic solvent (e.g., an amine), and sequential treatments such as a chemical treatment followed by washing. For instance, hydrogen peroxide can oxidize certain tungsten-containing deposits on the hydrogenation catalyst and can be followed by washing, especially with an aqueous alkaline solution or amine.

The treatment to enhance performance of the catalyst can be, or can include, a modulation of the activity of the Hcat such as a chemical treatment or deposit that attenuates catalytic sites. The treatment can also include a treatment with acidic or basic component to adjust the pH of the surface of the Hcat.

In one preferred aspect, continuous catalytic processes are provided for producing a lower glycol of at least one of ethylene glycol and propylene glycol from a carbohydrate-containing feed comprising at least one of aldose- and ketose-yielding carbohydrate, said processes being conducted in a reaction system containing a liquid medium wherein the reaction system contains homogeneous tungsten-containing retro aldol catalyst and at least a portion of the reaction system contains heterogeneous hydrogenation catalyst, said liquid medium being at catalytic conversion conditions including the presence of dissolved hydrogen, to produce a reaction product containing said glycol wherein tungsten-containing precipitate forms on the hydrogenation catalyst, wherein said processes comprise:
a. continuously or intermittently supplying the feed to the liquid medium in the reaction system;
b. maintaining the liquid medium in at least the portion of the reaction system containing hydrogenation catalyst under catalytic conversion conditions including the presence of dissolved hydrogen, to effect retro aldol and hydrogenation to produce a reaction product containing said glycol;
c. continuously or intermittently withdrawing a portion of the liquid medium containing reaction product from the reaction system; and
d. continuously or intermittently withdrawing a portion of the hydrogenation catalyst from the reaction system,
e. treating the withdrawn hydrogenation catalyst to remove at least a portion of the tungsten-containing deposit and provide a treated catalyst having enhanced performance; and
f. passing at least a portion of the treated catalyst to the reaction system.
   Preferably the treating of the hydrogenation catalyst comprises at least one of contacting the hydrogenation catalyst with exogenous carbonate anion and contacting the hydrogenation catalyst with an aqueous solution having a pH greater than about 5, say, from 6 to 11.

Another aspect pertains to conditioning of the heterogeneous nickel-containing catalyst ex situ. In accordance with this aspect, methods are provided for conditioning a heterogeneous, nickel-containing hydrogenation catalyst prior to use in a catalytic process for producing a lower glycol of at least one of ethylene glycol and propylene glycol from a carbohydrate-containing feed comprising at least one of aldose- and ketose-yielding carbohydrate, said process being conducted in a liquid medium containing a homogeneous tungsten-containing retro aldol catalyst and the heterogeneous nickel-containing hydrogenation catalyst, said method comprising contacting the hydrogenation catalyst with an aqueous medium containing water soluble salt of tungsten-containing anion and causing insoluble tungsten complexes or compounds to form and deposit on the hydrogenation catalyst to provide a hydrogenation catalyst having attenuated activity. Often, the deposits of insoluble tungsten complexes or compounds are effected at a pH below about 6 for a time sufficient to reduce the catalytic activity of the catalyst.

This disclosure also pertains to processes for attenuating the activity of a hydrogenation catalyst to be used in the conversion of carbohydrate feed to at least one of ethylene glycol and propylene glycol by a retro-aldol and hydrogenation process comprising contacting the hydrogenation catalyst with an aqueous solution of solubilized tungsten compound and then causing insoluble tungsten complexes or compounds to form and deposit on the hydrogenation catalyst to provide a hydrogenation catalyst having attenuated activity.

While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### DETAILED DESCRIPTION

All patents, published patent applications and articles referenced herein are hereby incorporated by reference in their entirety.

### Definitions

As used herein, the following terms have the meanings set forth below unless otherwise stated or clear from the context of their use.

Where ranges are used herein, the end points only of the ranges are stated so as to avoid having to set out at length and describe each and every value included in the range. Any appropriate intermediate value and range between the recited endpoints can be selected. By way of example, if a range of between 0.1 and 1.0 is recited, all intermediate values (e.g., 0.2, 0.3, 0.63, 0.815 and so forth) are included as are all intermediate ranges (e.g., 0.2-0.5, 0.54-0.913, and so forth).

The use of the terms “a” and “an” is intended to include one or more of the element described.

Admixing or admixed means the formation of a physical combination of two or more elements which may have a uniform or non-uniform composition throughout and includes, but is not limited to, solid mixtures, solutions and suspensions.

Aldose means a monosaccharide that contains only a single aldehyde group (—CH═O) per molecule and having the generic chemical formula Cₙ(H2O)ₙ. Non-limiting examples of aldoses include aldohexose (all six-carbon, aldehyde-containing sugars, including glucose, mannose, galactose, allose, altrose, idose, talose, and gulose); aldopentose (all five-carbon aldehyde containing sugars, including xylose, lyxose, ribose, and arabinose); aldotetrose (all four-carbon, aldehyde containing sugars, including erythrose and threose) and aldotriose (all three-carbon aldehyde containing sugars, including glyceraldehyde).

Aldose-yielding carbohydrate means an aldose or a di- or polysaccharide that can yield aldose upon hydrolysis. Sucrose, for example, is an aldose-yielding carbohydrate even though it also yields ketose upon hydrolysis.

Aqueous and aqueous medium or solution mean that water is present but does not require that water be the predominant component. For purposes of illustration and not in limitation, a solution of 90 volume percent of ethylene glycol and 10 volume percent water would be an aqueous solution. Aqueous solutions include liquid media containing dissolved or dispersed components such as, but not in limitation, colloidal suspensions and slurries.

Bio-sourced carbohydrate feedstock means a product that includes carbohydrates sourced, derived or synthesized from, in whole or in significant part, to biological products or renewable agricultural materials (including, but not limited to, plant, animal and marine materials) or forestry materials.

Catalyst for converting the carbohydrate means one or more catalysts to effect the catalytic conversion. For the hydrogenolysis route, catalyst for converting the carbohydrate would include mixtures of hydrogenolysis catalysts as well as a single hydrogenolysis catalyst. For the retro-aldol route, catalyst for converting the carbohydrate included both the retro-aldol catalyst and the hydrogenation catalyst, each of which can comprise one or a mixture of catalysts. The catalyst can contain one or more catalytic metals, and for Hcats, include supports, binders and other adjuvants. Catalytic metals are metals that are in their elemental state or are ionic or covalently bonded. The term catalytic metals refers to metals that are not necessarily in a catalytically active state, but when not in a catalytically active state, have the potential to become catalytically active. Catalytic metals can provide catalytic activity or modify catalytic activity such as promotors, selectivity modifiers, and the like.

Commencing contact means that a fluid starts a contact with a component, e.g., a medium containing a homogeneous or Hcat, but does not require that all molecules of that fluid contact the catalyst.

Compositions of aqueous solutions are determined using gas chromatography for lower boiling components, usually components having 3 or fewer carbons and a normal boiling point less than about 300° C., and high performance liquid chromatography for higher boiling components, usually 3 or more carbons, and those components that are thermally unstable.

Conversion efficiency of aldohexose to ethylene glycol is reported in mass percent and is calculated as the mass of ethylene glycol contained in the product solution divided by the mass of aldohexose theoretically provided by the carbohydrate feed and thus includes any aldohexose per se contained in the carbohydrate feed and the aldohexose theoretically generated upon hydrolysis of any di- or polysaccharide contained in the carbohydrate feed.

Hexitol means a six carbon compound having the empirical formula of C₆H₁₄O₆ with one hydroxyl per carbon.

High shear mixing involves providing a fluid traveling at a different velocity relative to an adjacent area which can be achieved through stationary or moving mechanical means to effect a shear to promote mixing. As used herein, the components being subjected to high shear mixing may be immiscible, partially immiscible or miscible.

Hydraulic distribution means the distribution of an aqueous solution in a vessel including contact with any catalyst contained therein.

Immediately prior to means no intervening unit operation requiring a residence time of more than one minute exists.

Intermittently means from time to time and may be at regular or irregular time intervals.

Ketose means a monosaccharide containing one ketone group per molecule. Non-limiting examples of ketoses include ketohexose (all six-carbon, ketone-containing sugars, including fructose, psicose, sorbose, and tagatose), ketopentose (all five-carbon ketone containing sugars, including xylulose and ribulose), ketotetrose (all four-carbon, ketose containing sugars, including erythrulose), and ketotriose (all three-carbon ketose containing sugars, including dihydroxyacetone).

Liquid medium means the liquid in the reactor. The liquid is a solvent for the carbohydrate, intermediates and products and for the homogeneous, tungsten-containing retro-aldol catalyst. Typically and preferably, the liquid contains at least some water and is thus termed an aqueous medium.

Lower glycol is ethylene glycol or propylene glycol or mixtures thereof.

The pH of an aqueous solution is determined at ambient pressure and temperature. In determining the pH of, for example the aqueous, hydrogenation medium or the product solution, the liquid is cooled and allowed to reside at ambient pressure and temperature for 2 hours before determination of the pH. Where the aqueous solution contains less than about 50 mass percent water, e.g., in a glycol-rich medium, water is added to a sample to provide a solution containing about 50 mass percent water. For purposes of consistency, the dilution of solutions is to the same mass percent water.

pH control agents means one or more of buffers and acids or bases.

A pressure sufficient to maintain at least partial hydration of a carbohydrate means that the pressure is sufficient to maintain sufficient water of hydration on the carbohydrate to retard caramelization. At temperatures above the boiling point of water, the pressure is sufficient to enable the water of hydration to be retained on the carbohydrate.

A rapid diffusional mixing is mixing where at least one of the two or more fluids to be mixed is finely divided to facilitate mass transfer to form a substantially uniform composition.

A reactor can be one or more vessels in series or in parallel and a vessel can contain one or more zones. A reactor can be of any suitable design for continuous operation including, but not limited to, tanks and pipe or tubular reactor and can have, if desired, fluid mixing capabilities. Types of reactors include, but are not limited to, laminar flow reactors, fixed bed reactors, slurry reactors, fluidized bed reactors, moving bed reactors, simulated moving bed reactors, trickle-bed reactors, bubble column and loop reactors.

Separation unit operations are one or more operations to selectively separate chemicals, including, but not limited to, chromatographic separation, sorption, membrane separation, flash separation, distillation, rectification, and evaporation.

Soluble means able to form a single liquid phase or to form a colloidal suspension.

Solubilized tungsten compounds are dissolved tungsten compounds or colloidally suspended tungsten compounds in the reaction medium.

Vapor/liquid separation is a separation providing one or more vapor streams and one or more liquid streams and can be based upon chromatographic separation, cyclic sorption, membrane separation, flash separation, distillation, rectification, and evaporation (e.g., thin film evaporators, falling film evaporators and wiped film evaporators).

### Carbohydrate Feed

The processes disclosed herein use a carbohydrate feed that contains an aldohexose-yielding carbohydrate or ketose-yielding carbohydrate, the former providing under retro-aldol reaction conditions, an ethylene glycol-rich product and the latter providing a propylene glycol-rich product. Where product solutions containing a high mass ratio of ethylene glycol to propylene glycol are sought, the carbohydrate in the feed comprises at least about 90, preferably at least about 95 or 99, mass percent of aldohexose-yielding carbohydrate. Often the carbohydrate feed comprises a carbohydrate polymer such as starch, cellulose, or partially to essentially fully hydrolyzed fractions of such polymers or mixtures of the polymers or mixtures of the polymers with partially hydrolyzed fractions.

The carbohydrate feed is most often at least one of pentose and hexose or compounds that yield pentose or hexose. Examples of pentose and hexose include xylose, lyxose, ribose, arabinose, xylulose, ribulose, glucose, mannose, galactose, allose, altrose, idose, talose, and gulose fructose, psicose, sorbose, and tagatose. Most bio-sourced carbohydrate feedstocks yield glucose upon being hydrolyzed. Glucose precursors include, but are not limited to, maltose, trehalose, cellobiose, kojibiose, nigerose, nigerose, isomaltose, β,β-trehalose, α,β-trehalose, sophorose, laminaribiose, gentiobiose, and mannobiose. Carbohydrate polymers and oligomers such as hemicellulose, partially hydrolyzed forms of hemicellulose, disaccharides such as sucrose, lactulose, lactose, turanose, maltulose, palatinose, gentiobiulose, melibiose, and melibiulose, or combinations thereof may be used.

The carbohydrate feed can be solid or, preferably, in a liquid suspension or dissolved in a solvent such as water. Where the carbohydrate feed is in a non-aqueous environment, it is preferred that the carbohydrate is at least partially hydrated. Non-aqueous solvents include alkanols, diols and polyols, ethers, or other suitable carbon compounds of 1 to 6 carbon atoms. Solvents include mixed solvents, especially mixed solvents containing water and one of the aforementioned non-aqueous solvents. Certain mixed solvents can have higher concentrations of dissolved hydrogen under the conditions of the hydrogenation reaction and thus reduce the potential for hydrogen starvation. Preferred non-aqueous solvents are those that can be hydrogen donors such as isopropanol. Often these hydrogen donor solvents have the hydroxyl group converted to a carbonyl when donating a hydrogen atom, which carbonyl can be reduced under the conditions in the reaction zone. Most preferably, the carbohydrate feed is provided in an aqueous solution. In any event, the volume of feed and the volume of raw product withdrawn need to balance to provide for a continuous process.

Further considerations in providing the carbohydrate to the reaction zone are minimizing energy and capital costs. For instance, in steady state operation, the solvent contained in the feed exits the reaction zone with the raw products and needs to be separated in order to recover the sought glycol products.

Preferably, the feed is introduced into the reaction zone in a manner such that undue concentrations of HOC's that can result in hydrogen starvation are avoided. With the use of a greater number of multiple locations for the supply of carbohydrate per unit volume of the reaction zone, the more concentrated the carbohydrate in the feed can be. In general, the mass ratio of water to carbohydrate in the carbohydrate feed is preferably in the range of 4:1 to 1:4. Aqueous solutions of 600 or more grams per liter of certain carbohydrates such as dextrose and sucrose are sometimes commercially available.

In some instances, recycled hydrogenation solution having a substantial absence of hydrogenation catalyst, or aliquot or separated portion thereof, is added as a component to the carbohydrate feed. The recycled hydrogenation solution can be one or more of a portion of the raw product stream or an internal recycle where hydrogenation catalyst is removed. Suitable solid separation techniques include, but are not limited to, filtration and density separation such as cyclones, vane separators, and centrifugation. With this recycle, the amount of fresh solvent for the feed is reduced, yet the carbohydrate is fed at a rate sufficient to maintain a high conversion per unit volume of reaction zone. The use of a recycle, especially where the recycle is an aliquot portion of the raw product stream, enables the supply of low concentrations of carbohydrate to the reaction zone while maintaining a high conversion of carbohydrate to ethylene glycol. Additionally, it is feasible to maintain the recycle stream at or near the temperature in the reaction zone and it as it contains tungsten-containing catalyst, retro-aldol conversion can occur prior to entry of the feed into the reaction zone. With the use of recycled hydrogenation solution, the mass ratio of carbohydrate to total recycled product stream and added solvent is often in the range of about 0.05:1 to 0.4:1, and sometimes from about 0.1:1 to 0.3:1. The recycled raw product stream is often from about 20 to 80 volume percent of the product stream.

The carbohydrate contained in the carbohydrate feed is provided at a rate of at least 50 or 100, and preferably, from about 150 to 500 grams per liter of reactor volume per hour. Optionally, a separate reaction zone can be used that contains retro-aldol catalyst with an essential absence of hydrogenation catalyst.

### The Conversion Process

In the processes, the carbohydrate feed is introduced into solvent that contains catalyst for the catalytic conversion and hydrogen. The solvent is frequently water but can be lower alcohol or polyalcohols of 1 to 6 carbons, especially methanol, ethanol, n-propanol and isopropanol. For the hydrogenolysis route, the catalyst is a hydrogenolysis catalyst, and for the retro-aldol route, a retro-aldol catalyst and hydrogenation catalyst.

### Hydrogenolysis Route

In the hydrogenolysis route, carbon-carbon bonds are cleaved by hydrogen using a hydrogenolysis catalyst under hydrogenolysis conditions. Typically, the carbohydrate feed is contacted with heterogeneous hydrogenolysis catalyst at elevated temperature in the presence of hydrogen to effect the hydrogenolysis and generate ethylene glycol and propylene glycol. The reaction temperatures can fall within a broad range, e.g., from about 120° C. to 300° C., but often temperatures below about 220° C., more particularly below about 200° C., to attenuate the production of 1,2-butanediol. The pressures (absolute) are typically in the range of about 15 to 300 bar (1500 to 30,000 kPa), say, from about 25 to 200 bar (2500 and 20000 kPa). The hydrogen partial pressure is typically in the range of about 15 to 200 bar (1500 to 20,000 kPa), say, from about 25 to 150 bar (2500 and 15000 kPa).

The hydrogenolysis reaction may be carried out in any suitable reactor, including, but not limited to, fixed bed, fluidized bed, trickle bed, moving bed, slurry bed, continuously stirred tank, loop reactors such as Buss Loop® reactors available from BUSS ChemTech AG, and structured bed. One type of reactor that can provide high hydrogen concentrations and rapid heating is cavitation reactor such as disclosed in U.S. Pat. No. 8,981,135 B2, herein incorporated by reference in its entirely.

The hydrogenolysis catalyst is frequently provided in an amount of from about 0.1 to 10, and more often, from about 0.5 to 5, grams per liter of liquid medium, and in a packed bed reactor the hydrogenation catalyst comprises about 20 to 80 volume percent of the reactor. The residence time of the aqueous phase in the reactor can vary over a wide range, and is usually from about 1 minute to 5 hours, say, from 5 to 200 minutes. In some instances, the weight hourly space velocity is from about 0.01 to 20 hr⁻¹ based upon total carbohydrate in the feed.

Heterogeneous hydrogenolysis catalysts can be supported and unsupported catalysts. Typical supports include, but are not limited to, silica, zirconia, ceria, titania, alumina, aluminosilicates, clays, carbon such as activated carbon, and magnesia. Hydrogenolysis metals include platinum, palladium, ruthenium, rhodium, iridium, nickel, copper, iron, and cobalt. The hydrogenolysis metals can be used alone or in combination with other hydrogenolysis metals or catalyst modifiers. Rhenium, molybdenum, vanadium, titanium, tungsten, and chromium have been suggested as modifiers. Usually the hydrogenolysis is promoted by base, which is often an alkali metal hydroxide or basic metal oxide. The pH is frequently in the range of about 6 to 9 or 12; however, hydrogenolysis can occur at higher and lower acidities.

### Retro-Aldol Route

In the retro-aldol route, the carbohydrate feed may or may not have been subjected to retro-aldol conditions prior to being introduced into the reaction zone, and the carbohydrate feed may or may not have been heated through the temperature zone of 170° C. to 230° C. upon contacting the liquid medium in the reaction zone. Thus, in some instances the retro-aldol reactions may not occur until the carbohydrate feed is introduced into the liquid medium, and in other instances, the retro-aldol reactions may have at least partially occurred prior to the introduction of the carbohydrate feed into the liquid medium in the reaction zone. It is generally preferred to quickly disperse the carbohydrate feed in the liquid medium especially where the hydrogenation medium is used to provide direct heat exchange to the carbohydrate feed. This dispersion can be achieved by any suitable procedure including, but not limited to, the use of mechanical and stationary mixers and rapid diffusional mixing. The use of multiple ports to introduce the feed into the reactor also facilitates quick dispersion.

The preferred temperatures for retro-aldol reactions are typically from about 230° C. to 300° C., and more preferably from about 240° C. to 280° C., although retro-aldol reactions can occur at lower temperatures, e.g., as low as 90° C. or 150° C. The pressures (absolute) are typically in the range of about 15 to 200 bar (1500 to 20,000 kPa), say, from about 25 to 150 bar (2500 and 15000 kPa). Retro-aldol reaction conditions include the presence of retro-aldol catalyst. A retro-aldol catalyst is a catalyst that catalyzes the retro-aldol reaction. Examples of compounds that can provide retro-aldol catalyst include, but are not limited to, heterogeneous and homogeneous catalysts, including catalyst supported on a carrier, comprising tungsten and its oxides, sulfates, phosphides, nitrides, carbides, halides, acids and the like. Tungsten carbide, soluble phosphotungstens, tungsten oxides supported on zirconia, alumina and alumina-silica are also included. Preferred catalysts are provided by soluble tungsten compounds and mixtures of tungsten compounds. Soluble tungstates include, but are not limited to, ammonium and alkali metal, e.g., sodium and potassium, paratungstate, partially neutralized tungstic acid, ammonium and alkali metal metatungstate and ammonium and alkali metal tungstate. Often the presence of ammonium cation results in the generation of amine by-products that are undesirable in the lower glycol product. Without wishing to be limited to theory, the species that exhibit the catalytic activity may or may not be the same as the soluble tungsten compounds introduced as a catalyst. Rather, a catalytically active species may be formed as a result of exposure to the retro-aldol reaction conditions. Tungsten-containing complexes are typically pH dependent. For instance, a solution containing sodium tungstate at a pH greater than 7 will generate sodium metatungstate when the pH is lowered. The form of the complexed tungstate anions is generally pH dependent. The rate that complexed anions formed from the condensation of tungstate anions are formed is influenced by the concentration of tungsten-containing anions. A preferred retro-aldol catalyst comprises ammonium or alkali metal tungstate that becomes partially neutralized with acid, preferably an organic acid of 1 to 6 carbons such as, but without limitation, formic acid, acetic acid, glycolic acid, and lactic acid. The partial neutralization is often from about 25 to 75%, i.e., on average from 25 to 75% of the cations of the tungstate become acid sites. The partial neutralization may occur prior to introducing the tungsten-containing compound into the reactor or with acid contained in the reactor.

The concentration of retro-aldol catalyst used may vary widely and will depend upon the activity of the catalyst and the other conditions of the retro-aldol reaction such as acidity, temperature and concentrations of carbohydrate. Typically, the retro-aldol catalyst is provided in an amount to provide from about 0.01 or 0.05 to 100, say, from about 0.02 or 0.1 to 50, grams of tungsten calculated as the elemental metal per liter of aqueous, hydrogenation medium. The retro-aldol catalyst can be added as a mixture with all or a portion of the carbohydrate feed or as a separate feed to the liquid medium or with recycling liquid medium or any combination thereof. Where the retro-aldol catalyst comprises two or more tungsten species and they may be fed to the reaction zone separately or together.

Frequently the carbohydrate feed is subjected to retro-aldol conditions prior to being introduced into the hydrogenation medium in the reaction zone containing hydrogenation catalyst. Preferably the introduction into the aqueous, hydrogenation medium occurs in less than one minute, and most often less than 10 seconds, from the commencement of subjecting the carbohydrate feed to the retro-aldol conditions. Some, or all of the retro-aldol reaction can occur in the reaction zone containing the hydrogenation catalyst. In any event, the most preferred processes are those having a short duration of time between the retro-aldol conversion and hydrogenation.

Under many process conditions useful in the disclosed processes, tungsten-containing precipitates can form and may be suspended or deposited on surfaces, including the surface of the hydrogenation catalyst where the activity of the hydrogenation catalyst can be affected.

The hydrogenation, that is, the addition of hydrogen atoms to an organic compound without cleaving carbon-to-carbon bonds, can be conducted at a temperature in the range of about 100° C. or 120° C. to 300° C. or more. Typically, the hydrogenation medium is maintained at a temperature of at least about 230° C. until substantially all carbohydrate is reacted to have the carbohydrate carbon-carbon bonds broken by the retro-aldol reaction, thereby enhancing selectivity to ethylene and propylene glycol. Thereafter, if desired, the temperature of the hydrogenation medium can be reduced. However, the hydrogenation proceeds rapidly at these higher temperatures. Thus, the temperatures for hydrogenation reactions are frequently from about 230° C. to 300° C., say, from about 240° C. to 280° C. Typically, in the retro-aldol process the pressures (absolute) are typically in the range of about 15 to 200 bar (1500 to 20,000 kPa), say, from about 25 to 150 bar (2500 and 15000 kPa). The hydrogenation reactions require the presence of hydrogen as well as hydrogenation catalyst. Hydrogen has low solubility in aqueous solutions. The concentration of hydrogen in the aqueous, hydrogenation medium is increased with increased partial pressure of hydrogen in the reaction zone. The pH of the aqueous, hydrogenation medium is often at least about 2.5 or 3, say, from about 3 or 3.5 to 8, and in some instances from about 3.5 or 4 to 7.5.

The hydrogenation is conducted in the presence of a hydrogenation catalyst. Frequently the hydrogenation catalyst is a supported, heterogeneous catalyst. It can be deployed in any suitable manner, including, but not limited to, fixed bed, fluidized bed, trickle bed, moving bed, slurry bed, loop bed, such as Buss Loop® reactors available from BUSS ChemTech AG, and structured bed. One type of reactor that can provide high hydrogen concentrations and rapid heating is cavitation reactor such as disclosed in U.S. Pat. No. 8,981,135 B2, herein incorporated by reference in its entirely. Cavitation reactors generate heat in localized regions and thus the temperature in these localized regions rather the bulk temperature of the liquid medium in the reaction zone is the temperature process parameter for purposes of this disclosure. Cavitation reactors are of interest for this process since the retro-aldol conversion can be very rapid at the temperatures that can be achieved in the cavitation reactor.

Nickel, ruthenium, palladium and platinum are among the more widely used reducing metal catalysts. However, many reducing catalysts will work in this application. The catalysts may be supported or unsupported such as Raney nickel. The reducing catalyst can be chosen from a wide variety of supported transition metal catalysts. One particularly favored catalyst for the reducing catalyst in this process is a supported, Ni—Re catalyst. A similar version of Ni/Re or Ni/Ir can be used with good selectivity for the conversion of the formed glycolaldehyde to ethylene glycol. Nickel-rhenium is a preferred reducing metal catalyst and may be supported on alumina, alumina-silica, silica or other supports. Supported Ni—Re catalysts with B as a promoter are useful. Generally, for slurry reactors, a supported hydrogenation catalyst is provided in an amount of less than 10, and sometimes less than about 5, say, about 0.1 or 0.5 to 3, grams per liter of nickel (calculated as elemental nickel) per liter of liquid medium in the reactor. As stated above, not all the nickel in the catalyst is in the zero-valence state, nor is all the nickel in the zero-valence state readily accessible by glycol aldehyde or hydrogen. Hence, for a particular hydrogenation catalyst, the optimal mass of nickel per liter of liquid medium will vary. For instance, Raney nickel catalysts would provide a much greater concentration of nickel per liter of liquid medium. Frequently in a slurry reactor, the hydrogenation catalyst is provided in an amount of at least about 5 or 10, and more often, from about 10 to 70 or 100, grams per liter of aqueous, hydrogenation medium and in a packed bed reactor the hydrogenation catalyst comprises about 20 to 80 volume percent of the reactor. In some instances, the weight hourly space velocity is from about 0.01 or 0.05 to 1 hr⁻¹ based upon total carbohydrate in the feed. Preferably the residence time is sufficient that glycol aldehyde and glucose are less than 0.1 mass percent of the reaction product, and most preferably are less than 0.001 mass percent of the reaction product.

The carbohydrate feed is at least 50 grams of carbohydrate per liter per hour, and is often in the range of about 100 to 700 or 1000, grams of carbohydrate per liter per hour.

In the disclosed processes, the combination of reaction conditions (e.g., temperature, hydrogen partial pressure, concentration of catalysts, hydraulic distribution, and residence time) are sufficient to convert at least about 95, often at least about 98, mass percent and sometimes essentially all of the carbohydrate that yields aldose or ketose. It is well within the skill of the artisan having the benefit of the disclosure herein to determine the set or sets of conditions that will provide the sought conversion of the carbohydrate.

### Treatment and Unit Operations

A portion of the Hcat is continuously or intermittently withdrawn from the reaction zone. The Hcat usually is contained in the liquid medium of the reaction zone and none or at least a portion of the liquid medium can be removed prior to a treatment unit operation. The withdrawn liquid medium containing Hcat can be processed by one or more unit operations to recover lower glycol, and then the Hcat processed by one or more unit operations, or the liquid medium containing Hcat can be withdrawn for only the purpose of treating the Hcat. The frequency and amount of Hcat withdrawn for treatment in one or more unit operations can be predetermined or can be in response to a change in process parameters such as a loss of hydrogenation or hydrogenolysis activity. Most Hcats are sufficiently robust that per 100 hours of operation, less than about 50, and often from about 10 to 40, mass percent of the Hcat is withdrawn. In some instances, as the Hcat ages, the frequency and amount of Hcat withdrawn is increased to reflect the underlying normal aging of the Hcat. The withdrawn Hcat can be continuously or intermittently subjected to one or more unit operations to enhance performance.

One unit operation for treating Hcat is to restore active catalyst sites through hydrogen treatment or even a reactivation. Although the catalytic conversion of carbohydrate to ethylene glycol and propylene glycol is conducted under reducing conditions, the presence of oxygenated moieties, especially in regions where the catalyst may be hydrogen starved, can result in some oxidation of catalytic metals and thus loss of hydrogenation activity. Typically, the rejuvenation by hydrogen is for a duration of from about 1 minute to 10 hours, say, from about 5 to 200 minutes. The temperature of the rejuvenation is often in the range of about 150° C. to 400° C. or more, and the hydrogen partial pressure is in the range of about 2000 to 20,000, e.g., 3000 to 10,000, kPa. Other techniques can be used to facilitate the rejuvenation or activation of the hydrogenation catalyst or hydrogenolysis catalyst alone or in combination with reducing with hydrogen. For instance, the catalyst can be treated with hydrazine or borohydride or subjected to oxidation, e.g., with oxygen or peroxide, before reduction or leaching.

Another unit operation for treating Hcat is washing or chemical removal of deposits. Any suitable liquid can be used for washing, and the washing may primarily remove deposits by physical forces or the liquid for washing can dissolve deposits. The liquid can be aqueous or non-aqueous. Advantageously, the liquid does not unduly leach the catalytically active metals. If the washing or chemical removal results in the oxidation of catalytic metal sites, it is usually preferred to subject the Hcat to reducing conditions or chemical treatment, e.g., with hydrazine, to activate catalytic sites.

The conditions of the washing or chemical removal can vary widely so long as the Hcat is not unduly adversely affected. The Hcat can be placed in a bath containing the liquid for washing or the liquid can be continuously or intermittently be passed over the catalyst. The ratio of liquid to catalyst being treated will vary depending upon the nature and amount of the deposits on the catalyst, the efficacy of the liquid and the conditions of the treatment, all as can be determined by one skilled in the art having the benefit of this disclosure. The temperature for the treatment is often from about 10° C. to 300° C., say, from about 20° C. to 280° C., and duration can range from 0.1 hour or less to over 100 hours. Unless a subsequent reducing treatment to activate the Hcat is used, it is preferred to conduct the treatment under an inert, and more preferably, a hydrogen atmosphere. Sometimes the absolute pressure is in the range of 500 to 20,000 kPa.

The reduction of tungsten-containing deposits on Hcats can be conducted using an aqueous solution having a pH greater than about 5, often, in the range of about 6 to 11 or more, and in some instances, in the range of about 7 to 9 or 10. The basicity can be maintained by any suitable means such as the addition of soluble hydroxide. One preferred aqueous solution contains exogenous carbonate anion. The exogenous carbonate anion may be supplied by any convenient means and form that will result in carbonate anion for contact with the hydrogenation catalyst. Usually, the exogenous carbonate ion is provided as one or more of carbonic acid, carbon dioxide and carbonate or bicarbonate of one or more of ammonium, alkali metal and alkaline earth metal cation, and preferably carbonate or bicarbonate of one or more of ammonium and alkali metal, especially sodium or potassium cation.

The concentration of carbonate anion in the aqueous medium contacting hydrogenation catalyst can vary widely. Those skilled in the art having the benefit of this disclosure will understand that the optimal concentrations will be dependent upon the sought result of the contacting, the condition of the hydrogenation catalyst, whether the contacting is continuous or intermittent, the conditions including pH and temperature and components and their concentrations of the aqueous medium, and the concentration of the hydrogenation catalyst in the aqueous medium during the contacting. In general, the carbonate anion is present in an amount (calculated as CO₃⁻²) of from about 0.01 to 500 grams per liter of aqueous solution. The duration of the contact can vary widely, e.g., from about 0.01 to 100 hours, and in some instances, from about 0.1 to 20 hours. The optimal duration of the contact will depend on the sought result and may change based upon the age of the hydrogenation catalyst. The intermittent contact can be a set periods of time or can be as required.

Organic solvents or co-solvents in an aqueous solution can find application. For instance, diols, particularly ethylene glycol and propylene glycol, have some ability to solubilize some insoluble tungsten-containing compounds and complexes such as tungstic acid. Other solvents include amines such as diethanol amine and triethanol amine. Organic solvents can facilitate removal of organic deposits on the Hcat. Since the removal of the deposits are ex-situ, solvents that may adversely affect the conversion of carbohydrate to lower glycols can find application provide that they are removed before returning the Hcat to the reactor. In some instances, subjecting the Hcat to reducing conditions to reactivate catalytic sites may be required.

Another unit operation is one or more steps to condition the Hcat. The conditioning modifies the performance of the Hcat. Conditioning includes, but is not limited to, loading the Hcat with hydrogen or placing on the Hcat one or more adjuvants that affect its performance such as adjusting the pH of the surface of the Hcat or providing promotors, modifiers, co-catalysts, or catalytic inhibitors. Conditioning can also include a selective poisoning or occlusion of catalytic sites to modulate catalytic active to balance, for example, the capacity for mass transfer of hydrogen to the Hcat with the activity of the Hcat. Hydrogen starvation can lead to the generation of acids and loss of conversion efficiency to lower glycols. Selective poisoning can be achieved by contact with an atmosphere containing a minor amount of oxygen or sulfide for a limited time to achieve the sought degree of conditioning. The conditioning can also be achieved by depositing organic or inorganic substance on the Hcat. The conditioning can serve to selectively restrict access to interior surfaces of the Hcat and/or can occlude a portion of the exterior catalyst sites to modulate catalytic activity. One such method is to contact the Hcat with an aqueous solution of solubilized tungsten compound and then reduce pH to cause insoluble tungsten complexes or compounds to form and deposit on the Hcat. The amount of deposition will depend, among other things, the reduced pH and time. Thus, the degree of occlusion can be controlled. Often, the deposition is at a temperature of from about 10° C. to 200° C. for a duration of from 0.01 to 10 hours. The pH is frequently reduced to from about 2 to 4.5, say, from 2 to 3.5. Since nickel oxide has some solubility in aqueous solutions at lower pH, the conditions used for the deposition should not unduly result in the loss of nickel.

Although the disclosure has been described with references to various embodiments, persons skilled in the art will recognized that changes may be made in form and detail without departing from the spirit and scope of this disclosure.

What is claimed is:

1. A catalytic process for producing a lower glycol comprising at least one of ethylene glycol and propylene glycol from a carbohydrate-containing feed that comprises at least one of aldose- and ketose-yielding carbohydrate, said process comprising continuously or intermittently supplying the feed to a reaction zone containing a liquid medium having therein one or more catalysts for converting said carbohydrate to said glycol, wherein at least one of the catalysts is a hydrogenolysis or hydrogenation catalyst that is suspended in the liquid medium, said liquid medium being at catalytic conversion conditions including the presence of dissolved hydrogen, to produce a reaction product containing said lower glycol, and continuously or intermittently withdrawing liquid medium that contains reaction product from the reaction zone, wherein
(i) continuously or intermittently at least a portion of said suspended catalyst is withdrawn from the reaction zone;
(ii) at least a portion of the withdrawn suspended catalyst is subjected to treatment to provide a treated catalyst having enhanced performance; and
(iii) at least a portion of the treated catalyst is passed to the reaction zone.

2. The process of claim 1 wherein the withdrawn catalyst has deposits and the treatment of the catalyst comprises washing or chemical removal of deposits.

3. The process of claim 2 wherein an aqueous liquid is used for the treatment.

4. The process of claim 3 wherein the aqueous liquid has a pH greater than 5.

5. The process of claim 4 wherein the aqueous liquid contains a base.

6. The process of claim 4 wherein the aqueous liquid comprises exogenous carbonate anion.

7. The process of claim 3 wherein the aqueous liquid contains lower glycol.

8. The process of claim 1 wherein the treatment of the catalyst comprises contact with hydrogen at a temperature in a range of about 150° C. to 400° C. and a hydrogen partial pressure in a range of about 2000 to 20,000 kPa for a time sufficient to effect reduction of the catalyst.

9. The process of claim 1 wherein the catalyst is treated with hydrazine or borohydride or subjected to oxidation.

10. The process of claim 1 wherein the treatment of the catalyst comprises a conditioning.

11. The process of claim 10 wherein the conditioning is placing on the catalyst an adjuvant that affects its performance.

12. The process of claim 11 wherein the adjuvant comprises at least one of promotors, modifiers, co-catalysts, or catalytic inhibitors.

13. The process of claim 10 wherein the conditioning comprises selective poisoning of the catalyst.

14. The process of claim 10 wherein the conditioning comprises depositing organic or inorganic substance on the catalyst.

15. The process of claim 14 wherein the catalyst is contacted with an aqueous solution of solubilized tungsten compound and causing insoluble tungsten complexes or compounds to form and deposit on the catalyst.

16. The process of claim 15 wherein the pH of the solution is reduced to cause insoluble tungsten complexes or compounds to form.

17. A continuous catalytic process for producing a lower glycol of at least one of ethylene glycol and propylene glycol from a carbohydrate-containing feed comprising at least one of aldose- and ketose-yielding carbohydrate, said processes being conducted in a reaction system containing a liquid medium wherein the reaction system contains homogeneous, tungsten-containing retro aldol catalyst and at least a portion of the reaction system contains heterogeneous hydrogenation catalyst, said liquid medium being at catalytic conversion conditions including the presence of dissolved hydrogen, to produce a reaction product containing said glycol wherein tungsten-containing precipitate forms on the hydrogenation catalyst, wherein said process comprises:
i. continuously or intermittently supplying the feed to the liquid medium in the reaction system;
ii. maintaining the liquid medium in at least the portion of the reaction system containing under catalytic conversion conditions including the presence hydrogenation catalyst and retro-aldol catalyst and the presence of dissolved hydrogen, to effect retro aldol and hydrogenation to produce a reaction product containing said glycol;
iii. continuously or intermittently withdrawing a portion of the liquid medium containing reaction product from the reaction system; and
iv. continuously or intermittently withdrawing a portion of the hydrogenation catalyst from the reaction system,
v. treating the withdrawn hydrogenation catalyst to remove at least a portion of the tungsten-containing deposit and provide a treated catalyst having enhanced performance; and
vi. passing at least a portion of the treated catalyst to the reaction system.

18. The process of claim 17 wherein the hydrogenation catalyst is a nickel-containing hydrogenation catalyst.

19. The process of claim 17 wherein the treatment of the catalyst comprises washing or chemical removal of deposits.

20. A process for attenuating the activity of a hydrogenation catalyst for use in the conversion of carbohydrate feed to at least one of ethylene glycol and propylene glycol by a retro-aldol and hydrogenation process comprising contacting the hydrogenation catalyst with an aqueous solution of solubilized tungsten compound and causing insoluble tungsten complexes or compounds to form and deposit on the hydrogenation catalyst to provide a hydrogenation catalyst having attenuated activity.

21. The process of claim 20 wherein the hydrogenation catalyst is a nickel-containing hydrogenation catalyst.

22. The process of claim 21 wherein the pH of the solution is reduced to cause insoluble tungsten complexes or compounds to form.
